(19) United States
(12) Patent Application Publication
(10) Pub. No.: US 2026/0256610 A1
(43) Pub. Date: Sep. 3, 2026

(54) MALE EXTERNAL CATHETER WITH ATTACHMENT INTERFACE CONFIGURED TO BIAS AGAINST PENIS

(71) Applicant: PureWick Corporation, Covington, GA (US)

(72) Inventors: Camille Rose Newton, Bonsall, CA (US); Raymond John Newton, Bonsall, CA (US); Benjamin Jackson, Statham, GA (US)

(21) Appl. No.: 19/544,501

(22) Filed: Feb. 19, 2026

Related U.S. Application Data
(63) Continuation of application No. 17/645,821, filed on Dec. 23, 2021, now Pat. No. 12,599,495.
(60) Provisional application No. 63/133,892, filed on Jan. 5, 2021.

(51) Int. Cl. A61F 5/453; A61F 5/44; A61F 5/443

(52) U.S. Cl. CPC A61F 5/453; A61F 5/4404; A61F 5/443

(57) ABSTRACT

An example male urine collection device for collection of urine discharged from a subject is disclosed. The male urine collection device includes a receptacle defining an internal volume, the receptacle configured to receive a penis and to collect urine, and a plurality of flaps movable to access the internal volume of the receptacle, the flaps configured to bias against the penis when the penis is at least partially received by the receptacle. In an embodiment, a male urine collection device includes a receptacle defining an internal volume, the receptacle configured to collect urine, and a clamshell having two halves movable with respect to each other via a hinge, the clamshell configured to close around the penis to secure the receptacle about the penis. The clamshell is removably secured to the penis via friction.

### CROSS-REFERENCE TO RELATED APPLICATIONS

This application is a continuation of U.S. application Ser. No. 17/645,821 filed on Dec. 23, 2021, which is claims priority to U.S. Provisional Application No. 63/133,892 filed on Jan. 5, 2021, the disclosure of each of which is incorporated herein, in its entirety, by this reference.

### BACKGROUND

In various circumstances, a person or animal may have limited or impaired mobility such that typical urination processes are challenging or impossible. For example, a person may experience or have a disability that impairs mobility. A person may have restricted travel conditions such as those experienced by pilots, drivers, and workers in hazardous areas. Additionally, sometimes urine collection is needed for monitoring purposes or clinical testing.

Urinary catheters, such as a Foley catheter, can be used to address some of these circumstances, such as incontinence. Unfortunately, however, urinary catheters can be uncomfortable, painful, and can lead to complications, such as infections. Additionally, bed pans, which are receptacles used for the toileting of bedridden patients, such as those in a health care facility, are sometimes used. Bed pans, however, can be prone to discomfort, spills, and other hygiene issues. Males who can suffer severe consequences of urinary incontinence, such as discomfort, rashes, and sores are typically elderly and often bedbound. They also require continuous assistance to maintain hygiene. Characteristics often found in these patients: they typically lay on their back, the size of the penis often decreases with age, skin rolls containing fat tissue cause the penis to recede, often pointing upward while in a laying position, patients have difficulty reaching the penis and manipulating devices.

### SUMMARY

Embodiments for attachment interfaces and methodologies disclosed herein improve upon conventional devices and methods for attaching urine collection devices to a subject's penis. The devices disclosed herein enable improved attachment to the penis while still allowing for easy removal without injury or irritation to the subject. In an embodiment, a male urine collection device for collection of urine discharged from a subject is disclosed. The male urine collection device includes a receptacle defining an internal volume, the receptacle configured to receive a penis and to collect urine, and a plurality of flaps movable to access the internal volume of the receptacle. The plurality of flaps are configured to bias against the penis when the penis is at least partially received by the receptacle.

In an embodiment, a male urine collection device for collection of urine discharged from a penis is disclosed. The male urine collection device includes a receptacle defining an internal volume, the receptacle configured to collect urine, and a clamshell having two halves movable with respect to each other via a hinge, the clamshell configured to close around the penis to secure the receptacle about the penis when the penis is at least partially received by the receptacle. The clamshell is removably secured to the penis via friction.

Features from any of the disclosed embodiments may be used in combination with one another, without limitation. In addition, other features and advantages of the present disclosure will become apparent to those of ordinary skill in the art through consideration of the following detailed description and the accompanying drawings.

### BRIEF DESCRIPTION OF THE DRAWINGS

The drawings illustrate several embodiments of the present disclosure, wherein identical reference numerals refer to identical or similar elements or features in different views or embodiments shown in the drawings.

FIGS. 1A and 1B are top and bottom isometric views, respectively, of a male urine collection device, according to an embodiment.

FIG. 2A is an isometric view of a male urine collection device, according to an embodiment.

FIG. 2B is a cross-sectional side view of the male urine collection device of FIG. 2A.

FIG. 3A is a top view of a urine collection device, according to an embodiment.

FIG. 3B is a cross-sectional side view of the urine collection device of FIG. 3A.

FIG. 4A is an isometric view of a urine collection device, according to an embodiment.

FIG. 4B is a top view of an attachment interface, according to an embodiment.

FIG. 5 is a top view of an attachment interface, according to an embodiment.

FIG. 6 is a top view of an attachment interface, according to an embodiment.

FIG. 7 is an isometric view of a bandage, according to an embodiment.

FIG. 8 is an isometric view of the bandage of FIG. 5 coupled to a urine collection device, according to an embodiment.

FIG. 9 is a top view of an attachment interface, according to an embodiment.

FIG. 10 is a bottom isometric view of a urine collection device, according to an embodiment.

FIG. 11 is a bottom isometric view of a urine collection device having both the attachment interfaces of FIGS. 4B and 7, according to an embodiment.

FIG. 12A is a side view of a securement feature, according to an embodiment.

FIG. 12B is a side view of a securement feature, according to an embodiment.

FIG. 12C is an isometric view of the securement feature of FIG. 11B in an engaged state, according to an embodiment.

FIG. 13 is a bottom isometric view of a urine collection device having the attachment interfaces of FIGS. 4B and 7 and the securement feature of FIG. 11B, according to an embodiment.

FIG. 14 is a schematic illustration of a urine collection device disposed on the body of a user, according to an embodiment.

### DETAILED DESCRIPTION

Embodiments for attachment interfaces and methodologies disclosed herein improve upon conventional devices and methods for attaching urine collection devices, such as male external catheters, to a subject's penis. The devices disclosed herein enable improved attachment to the penis while still allowing for easy removal without injury or irritation to the subject. A urine collection device is disclosed that is suitable for collecting and transporting urine away from the body of a person or other animal, particularly a male. The disclosed urine collection device includes a urine collecting assembly that can include an attachment interface for improved functionality and securement to the penis.

In some embodiments, the attachment interface includes a plurality of flaps that can surround the penis to removably secure to the penis. In some embodiments, the attachment interface can include a clamshell device configured to lightly clamp onto the penis. The urine collection device can further include a fluid receptacle configured to collect urine from a subject. The attachment interfaces described herein can at least partially define the receptacle. For example, the attachment interface can define one or more walls of the receptacle. The receptacle also includes a fluid outlet in fluid communication with an outlet tube that may be, for example, in fluid communication with a vacuum pump. The urine collection device can be configured to receive a user's penis such that the urethral opening of the penis is disposed within the receptacle (e.g., disposed within the interior region of the receptacle) and the shaft of the penis is in sealing relationship with a peripheral edge of an opening defined by the attachment interface such that the urine collection device is configured to receive urine discharged from the urethral opening into the receptacle, and to have the received urine withdrawn from the receptacle via the outlet.

Examples of male urine collection devices that are configured to collected bodily fluids from a male urethral opening and methods of using such fluid collection assemblies are disclosed in International Application No. PCT/US 2020/42262 filed on Jul. 14, 2020, U.S. Patent Publication No. 2019/0282391 filed on Jun. 6, 2019, U.S. Pat. No. 10,376,406 patented on Aug. 13, 2019, and U.S. Provisional Patent Application No. 63/067,542 filed on Aug. 19, 2020, the disclosure of each of which is incorporated herein, in its entirety, by this reference. Other embodiments of fluid impermeable barriers, fluid permeable membranes, fluid permeable supports, chambers, and their shapes and configurations are disclosed in U.S. patent application Ser. No. 15/612,325 filed on Jun. 2, 2017; U.S. patent application Ser. No. 15/260,103 filed on Sep. 8, 2016; and U.S. Pat. No. 10,225,376 filed on Jun. 1, 2017, the disclosure of each of which is incorporated herein, in its entirety, by this reference.

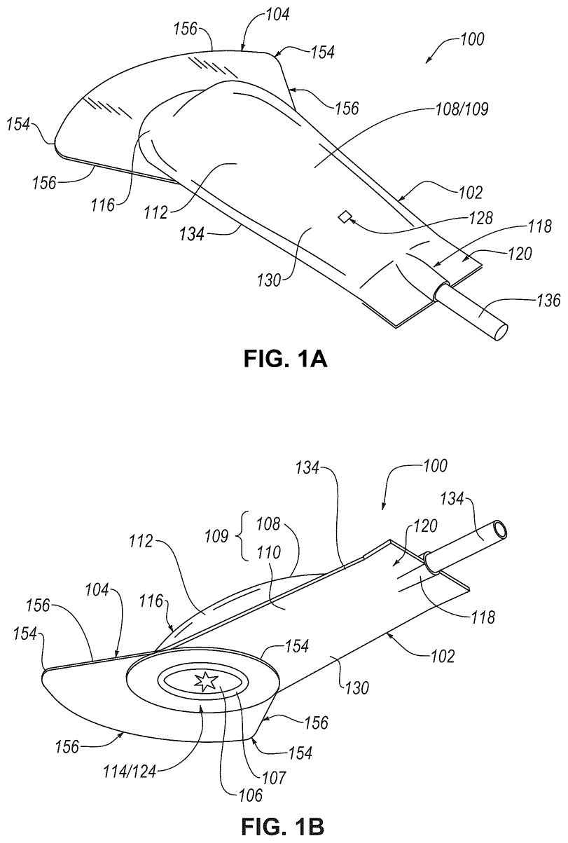

FIGS. 1A and 1B are isometric top and bottom views, respectively, of a urine collection device 100, according to an embodiment. The urine collection device 100 includes a sheath 102 and a base 104. The sheath 102 includes a fluid impermeable barrier 109 that is at least partially formed from a first panel 108 attached to a second panel 110. In an embodiment, as illustrated, the first panel 108 and the second panel 110 are distinct sheets. The fluid impermeable barrier 109 also defines a chamber 112 between the first panel 108 and the second panel 110, an opening 114 at a proximal end region 116 of the sheath 102, and an outlet 118 at a distal end region 120 of the sheath 102. The sheath 102 also includes at least one porous material disposed in the chamber 112. The base 104 includes an aperture 124. The base 104 is permanently attached to the proximal end region 116 of the sheath 102 such that the aperture 124 is aligned with the opening 114. Permanently attached means that the sheath 102 cannot be detached from the base 104 without damaging at least one of the sheath 102 or the base 104, using a blade to separate the sheath 102 from the base 104, and/or using chemicals to dissolve the adhesive that attaches the sheath 102 from the base 104.

The inner surfaces 126 of the fluid impermeable barrier 109 (e.g., inner surfaces of the first and second panels 108, 110) at least partially defines the chamber 112 within the urine collection device 100. The fluid impermeable barrier 109 temporarily stores the bodily fluids in the chamber 112. The fluid impermeable barrier 109 may be formed of any suitable fluid impermeable material(s), such as a fluid impermeable polymer (e.g., silicone, polypropylene, polyethylene, polyethylene terephthalate, a polycarbonate, etc.), a metal film, natural rubber, another suitable material, or combinations thereof. As such, the fluid impermeable barrier 109 substantially prevents the bodily fluids from passing through the fluid impermeable barrier 109. In an example, the fluid impermeable barrier 109 may be air permeable and fluid impermeable thus preventing leaks while allowing air flow through the chamber 112 when a suction force is applied thereto (i.e., the chamber 112 remains at about atmospheric pressure thereby preventing the suction force from causing a bruise or kinking the conduit 136). In such an example, the fluid impermeable barrier 109 may be formed of a hydrophobic material that defines a plurality of pores. Alternatively or additionally, the fluid impermeable barrier 109 may include at least one perforation 128 (e.g., vacuum relief hole) that allows the chamber 112 to remain substantially at atmospheric pressure. At least one or more portions of at least an outer surface 130 of the fluid impermeable barrier 109 may be formed from a soft and/or smooth material, thereby reducing chaffing.

In an embodiment, at least one of the first panel 108 or the second panel 110 is formed from an at least partially transparent fluid impermeable material, such as polyethylene, polypropylene, polycarbonate, or polyvinyl chloride. Forming at least one of the first panel 108 or the second panel 110 from an at least partially transparent fluid impermeable material allows a person (e.g., medical practitioner) to examiner the penis. In some embodiments, both the first panel 108 and the second panel 110 are formed from at least partially transparent fluid impermeable material. For example, some conventional fluid collection assemblies that include a sheath and a base may allow the sheath to be reversibly detached from the base after the base is secured to the region about the penis. Detaching the sheath from the base allows the person to examine the penis. However, configuring the sheath to be detachable from the base may allow leaks between the sheath and the base. As previously discussed, the sheath 102 is permanently attached to the base 104 which substantially prevents leaks between the sheath 102 and the base 104 when the base 104 is appropriately attached to the sheath 102 (e.g., no wrinkles were allowed to form between the sheath 102 and base 104). Selecting at least one of the first panel 108 or the second panel 110 to be formed from an at least partially transparent impermeable material allows the penis to be examined without detaching the entire urine collection device 100 from the region about the penis. For example, the chamber 112 may include a penis receiving area 132 that is configured to receive the penis of the individual when the penis extends into the chamber 112.

The porous material in the chamber 112 may include permeable material designed to wick or pass fluid therethrough. Suitable permeable or porous materials can include spun nylon fibers. The permeable properties referred to herein may be wicking, capillary action, diffusion, or other similar properties or processes, and are referred to herein as “permeable” and/or “wicking.” Such “wicking” may not include absorption of fluid into the wicking material. Put another way, substantially no absorption of fluid into the material may take place after the material is exposed to the fluid and removed from the fluid for a time. While no absorption is desired, the term “substantially no absorption” may allow for nominal amounts of absorption of fluid into the wicking material (e.g., absorbency), such as less than about 30 wt % of the dry weight of the wicking material, less than about 20 wt %, less than about 10 wt %, less than about 7 wt %, less than about 5 wt %, less than about 2 wt %, or less than about 0.5 wt % of the dry weight of the wicking material. Wicking material can include natural fibers. In such examples, the material may have a coating to prevent or limit absorption of fluid into the material, such as a water repellent coating. In an embodiment, the porous material includes a body of spun nylon fibers with an outer permeable membrane made of fabric such as gauze.

The penis receiving area 132 may be defined by at least the porous material and at least a portion of the at least partially transparent material of the first panel 108 and/or the second panel 110. In other words, the porous material is positioned in the chamber 112 such that the porous material is not positioned between the penis and at least a portion of the transparent portion of the first panel 108 and/or second panel 110 when the penis is inserted into the chamber 112 through the opening 114. The porous material is generally not transparent and, thus, the portion of the at least partially transparent material of the first panel 108 and/or the second panel 110 that defines the penis receiving area 132 forms a window which allows the person to view into the penis receiving area 132 and examine the penis.

In an embodiment, the second panel 110 is at least partially formed from the at least partially transparent material and forms the window that allows the person to view into the penis receiving area 132. Further, the porous material is positioned between the penis receiving area 132 and at least a portion of the first panel 108. Such an embodiment may help maintain the dignity of the individual using the urine collection device 100. For example, during use, the second panel 110 is generally adjacent to the individual, such as adjacent to the thighs and/or perineum. Thus, the second panel 110 is generally obscured during use and a person cannot view the penis without first lifting the sheath 102 away from the individual. Meanwhile, the first panel 108 may face away from the individual and be more easily viewable than the second panel 110. However, a person (e.g., a passerby, a visitor, etc.) cannot view the penis through the first panel 108 because the porous material is not transparent and/or the first panel 108 is formed from a non-transparent material. Thus, in such an embodiment, the first panel 108 and/or the porous material prevent person(s) from viewing the penis unless such examination is necessary, thereby preserving the dignity of the individual using the urine collection device 100. In an embodiment, the first panel 108 is formed from the at least partially transparent material and forms the window that allows the person to view into the penis receiving area 132. Further, the porous material is positioned between the penis receiving area 132 and at least a portion of the second panel 110. In such an embodiment, the person does not need to perform the additional act of lifting the sheath 102 to view into the penis receiving area 132 but may not maintain the dignity of the individual using the urine collection device 100 since passersby may also view into the penis receiving area 132.

As previously discussed, at least a portion of the first panel 108 and at least a portion of the second panel 110 are attached together. In an embodiment, as shown, the first and second panels 108, 110 are attached together along at least a portion of the outer edges 134 thereof. In such an embodiment, the first and second panels 108, 110 are attached using any suitable technique, such as with an adhesive, sewing, heat sealing, radio frequency (“RF”) welding, ultrasonic (“US”) welding, or any other technique. As will be discussed in more detail below, forming the fluid impermeable barrier 109 from the first panel 108 and the second panel 110 may improve the rate of manufacturing the urine collection device 100, especially when the first panel 108 and the second panel 110 are attached together using a non-sewing technique. In an embodiment, the first panel 108 and the second panel 110 can be a single unitary piece, such as a tube.

The opening 114 defined by the fluid impermeable barrier 109 provides an ingress route for fluids to enter the chamber 112 when the penis is a buried penis and allow the penis to enter the chamber 112 (e.g., the penis receiving area 132) when the penis is not buried. The opening 114 may be defined by the fluid impermeable barrier 109 (e.g., an inner edge of the fluid impermeable barrier 109). For example, the opening 114 is formed in and extends through the fluid impermeable barrier 109, from the outer surface 130 to the inner surface 126, thereby enabling bodily fluids to enter the chamber 112 from outside of the urine collection device 100.

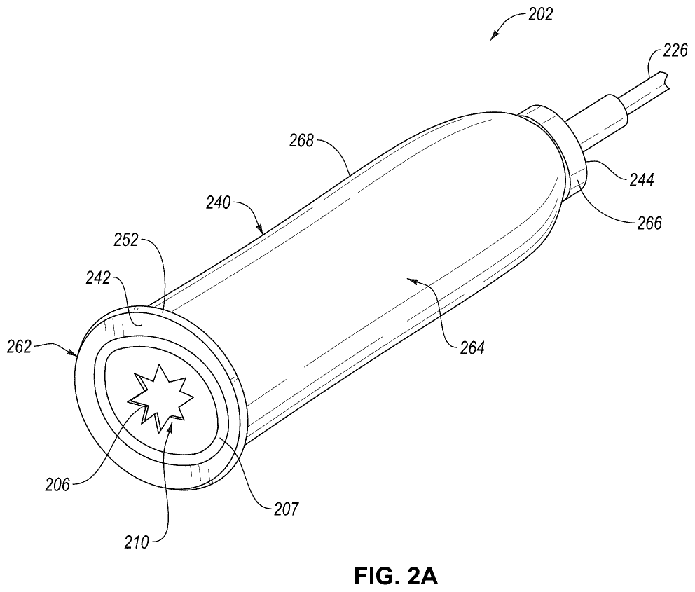

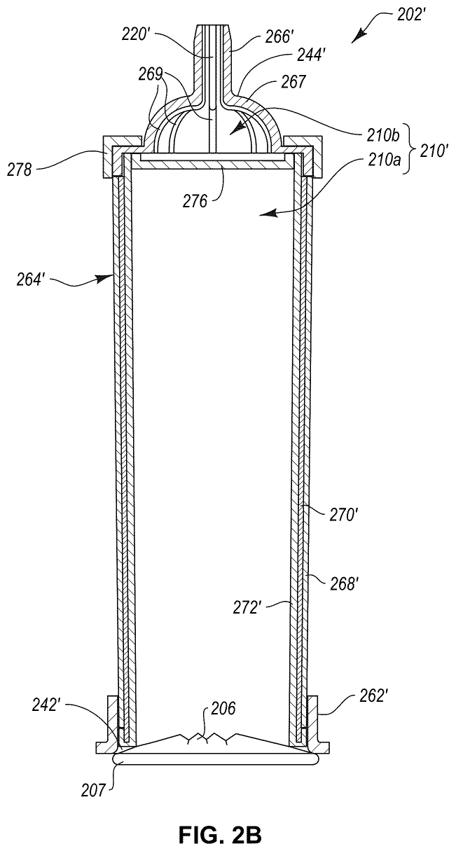

In an embodiment, the urine collection device 100 includes one or more attachment interfaces 106, 107 disposed in or about the opening 114. The attachment interfaces 106, 107 can be the same or similar to any of the attachment interfaces disclosed herein, for example, the attachment interface 106 can be substantially similar to the attachment interface 306 and the attachment interface 107 (also referred to as a clamshell), can be substantially similar to the attachment interface 407 as described below. As discussed below, the attachment interfaces 106, 107 can be used to provide improve attachment of the urine collection device 100 to the penis of the subject. It will be appreciated that the attachment interfaces 106, 107 can be used with a wide variety of urine collection devices and the embodiment shown in FIG. 1B is non-limiting. Further examples of attachment interfaces being implemented on urine collection devices are provided below with reference to FIGS. 2A and 2B.

FIG. 2A is an isometric view of a urine collection device 202, according to an embodiment. The urine collection device 202 can be similar to any of the urine collection devices disclosed herein. For example, the urine collection device 202 can include a body 240 having an open proximal end 242 and an at least partially closed distal end 244, a reservoir 210 at least partially defined by the body 240, attachment interfaces 206, 207, and tubing 226 fluidly coupled to the reservoir 210.

The body 240 of the urine collection device 202 can include a ring 262 at or near the open proximal end 242 of the body 240, a sheath 264 extend from or near the open proximal end 242 to or near the at least partially closed distal end 244 of the body 240, and a sump 266 at the at least partially closed distal end 244 of the body 240. In an embodiment, the ring 262, the sheath 264, and the sump 266 are all distinct components from each other. In an embodiment, at least two of the ring 262, the sheath 264, or the sump 266 are integrally formed together (e.g., are formed from a single piece). The sheath 264 is configured to prevent a fluid (e.g., urine) escaping from the reservoir 210 and to move the fluid towards the sump 266 and the tubing 226. The sheath 264 can include a plurality of layers that facilitate the operation of the sheath 264.

The urine collection device 202 can include attachment interfaces 206, 207 located at or near the open proximal end 242 of the body 240. The attachment interfaces 206, 207 can be similar to the attachment interfaces discussed herein, for example, the attachment interface 206 can be substantially similar to the attachment interface 306 and the attachment interface 207 (also referred to as a clamshell), can be substantially similar to the attachment interface 407 as described below. A subject's penis may enter the body 240 of the urine collection device 202 by passing the attachment interfaces 206, 207. The urine collection device 202 may then be securely affixed to the subject's penis via the flaps of the attachment interface 206 and clamshell 207 as discussed herein. Further details of attachment interfaces being incorporated onto urine collection devices are discussed below. Specifically, the remaining figures disclose structural and functional details of the attachment interfaces and highlight the applicability of the attachment interfaces to generic urine collection devices.

FIG. 2B is a cross-sectional side view of a urine collection device 202, according to an embodiment. For example, with reference to FIG. 2B the sheath 264 can include a fluid impermeable layer 268, a fluid permeable layer (e.g., a one-way fluid movement fabric, gauze, or cloth), and a fluid permeable support 272 positioned between the fluid permeable layer and the fluid impermeable layer 268. The fluid permeable support 272 may include a porous layer (e.g., a spun polymer layer. The fluid impermeable layer 268 can form an external surface of the body 240 and prevent the fluid from leaking through the sheath 264. The fluid permeable layer 274 can form an internal surface of the body 240. The fluid permeable layer 274 can be configured to move the fluid from the reservoir 210 to the fluid permeable support 272 and may substantially prevent the fluid that is in the fluid permeable support 272 from flowing back into the reservoir 210. As such, the fluid permeable layer 274 can remove fluid from around a penis thereby leaving the penis dry. The fluid permeable support 272 can form an inner layer between the fluid permeable layer 274 and the fluid impermeable layer 268. The fluid permeable support 272 can enable the fluid to flow generally towards the tubing 226.

It is noted that one or more layers of the sheath 264 can be omitted. For example, the fluid permeable layer 274 can be omitted such that the fluid permeable support 272 forms the internal surface of the body 240. In such an example, the sheath 264 can rely on the wicking ability of the fluid permeable support 272 and a suction force applied to the urine collection device 202 to remove the fluid from the penis. In another example, the sheath 264 only include the fluid impermeable layer 268. In such an example, the sheath 264 can rely on the suction force applied to the urine collection device 202 to remove the fluid from the penis. In another example, the sheath 264 only includes the fluid impermeable layer 268 and the fluid permeable layer 274. In such an example, the sheath 264 can form a channel (not shown) between the fluid impermeable layer 268 and the fluid permeable layer 274 and the channel is fluidly coupled to the tubing 226.

The fluid permeable layer 274 and/or the fluid permeable support 272 may include permeable material designed to wick or pass fluid therethrough. The permeable properties referred to herein may be wicking, capillary action, diffusion, or other similar properties or processes, and are referred to herein as “permeable” and/or “wicking.” Such “wicking” may not include absorption of fluid into the wicking material. Put another way, substantially no absorption of fluid into the material may take place after the material is exposed to the fluid and removed from the fluid for a time. While no absorption is desired, the term “substantially no absorption” may allow for nominal amounts of absorption of fluid into the wicking material (e.g., absorbency), such as less than about 10 wt % of the dry weight of the wicking material, less than about 7 wt %, less than about 5 wt %, less than about 3 wt %, less than about 2 wt %, less than about 1 wt %, or less than about 0.5 wt % of the dry weight of the wicking material. Wicking material can include natural fibers. In such examples, the material may have a coating to prevent or limit absorption of fluid into the material, such as a water repellent coating.

The sheath 264 is configured to have a penis disposed therein. To facilitate fluid collection and improve comfort, the sheath 264 can be flexible thereby allowing the sheath 264 to correspond to the shape of a penis. For example, the flexible sheath 264 can at least partially collapse when the penis is not erect and at least partially expand and bend to the shape of the penis as the penis becomes erect. Forming the layers of the sheath 264 from at least one of thin layers (e.g., less than 500 μm thick, and more particularly less than 250 μm thick, less than 100 μm thick, or less than 50 μm thick), flexible layers, or fabric can allow the sheath 264 to be sufficiently flexible.

The ring 262 can be more rigid than the sheath 264. For example, the ring 262 can be formed from a flexible polymer that is at least one of thicker than the entire sheath 264 or exhibits a Young's modulus that is greater than sheath 264. As such, the ring 262 can provide some structure at or near the open proximal end 242. The increased rigidity of the ring 262 can cause the open proximal end 242 to remain open thereby facilitating insertion of a penis into the attachment interface 206 and/or clamshell 207. Further, in an embodiment, the increased rigidity of the ring 262 can enable the ring 262 to act as an attachment mechanism. For example, as illustrated, the ring 262 can be adjacent to or enable placement of the attachment interface 206 and/or the clamshell 207.

The sump 266 is configured to attach the rest of the urine collection device 202 to the tube 226. For example, the sump 266 can define an outlet 220 extending through at least the fluid impermeable layer 268 thereby coupling the tubing 226 to the fluid permeable support 272 and/or the reservoir 210. Further, the sump 266 can close the at least partially closed distal end 244. For example, the sump 266 can bunch up the sheath 264 and close any gaps that may form.

The ring 262, the sheath 264, the sump 266, and attachment interface 206, 200 can be attached together using any suitable method. For example, at least two of the ring 262, the sheath 264, the sump 266, or attachment interfaces 206, 207 can be attached together using at least one of an interference fit, an adhesive, stitching, welding (e.g., ultrasonic welding), tape, any other suitable method, or combinations thereof.

The reservoir 210 may include one or more sections. For example, the reservoir 210 may include a first section 210a that is defined by the sheath 264. However, as illustrated, the reservoir 210 may include one or more additional sections that are defined by one or more additional components of the urine collection device 202. For example, sump 266 may exhibit a bulbous portion 267 extending from an outlet 220 of the sump 266 towards an open proximal end 242 of the sheath 264. The bulbous portion 267 may define a second section 210b of the reservoir 210. The second section 210b may provide a location to store the fluid that is spaced from a penis of a user that is partially disposed in the urine collection device 202. As such, the second section 210b may decrease the amount of fluids that contact the penis.

The sheath 264 extends from or near an open proximal end 242 of the urine collection device 202 to or near an at least partially closed distal end 244 of the urine collection device 202. However, in an embodiment, the portion of the sheath 264 at or near the at least partially closed distal end 244 defines a hole (e.g., opening). In such an embodiment, the urine collection device 202 may include a porous top layer 276 that at least partially covers the hole. The porous top layer 276 may separate the first section 210a of the reservoir 210 from the second section 210b of the reservoir 210.

The porous top layer 276 may be the same as or substantially similar to any of the fluid impermeable supports or fluid permeable layers disclosed herein. For example, the porous top layer 276 may include a one-way fluid movement fabric, thereby allowing fluid to flow from the first section 210a of the reservoir 210 to the second section 210b of the reservoir 210 while preventing the fluid from flowing from the second section 210b back to the first section 210a. As such, the porous top layer 276 may decrease the amount of fluid that contacts the penis when the porous top layer 276 includes the one-way fluid movement fabric. However, it is noted that the porous top layer 276 may include gauze, other types of fabric, or any other suitable porous material instead of or in addition to the one-way fluid movement fabric.

The sump 266 includes a bulbous portion 267. The sump 266 may also include one or more ridges 269 extending from or near the outlet 220 towards the open proximal end 242. In an embodiment, the ridges 269 may direct any fluids that are in the second section 210b of the reservoir 210 towards the outlet 220. In an embodiment, the ridges 269 may provide at least some structural support to the bulbous portion 267 such that ridges 269 resist the deformation and/or collapse of the bulbous portion 267. It is noted that the bulbous portion 267 may include one or more structural elements instead of or in addition to the ridges 269. For example, the bulbous portion 267 may define one or more recesses that resist bending stresses.

The sump 266 may be exposed to the atmosphere, which allows the sump 266 to be visible, and may be exposed to an interior of the urine collection device 202. As such, the sump 266 may provide fluid communication between the atmosphere and the interior of the urine collection device 202 and/or provide information. For example, the sump 266 may be formed from a porous material that is configured to assist in suction balance. In an embodiment, the sump 266 may be formed from a material that is configured to change color or state when exposed to bacteria to indicate that an individual using the urine collection device 202 has an infection.

The urine collection device 202 may include an upper ring 278. The upper ring 278 may be configured to be positioned adjacent to the portion of the sump 266 that is connected to the sheath 264. In an embodiment, the upper ring 278 provides additional strength and/or rigidity to the portion of the sump 266 that is connected to the sheath 264. For example, the sheath 264 may be flexible, thereby allowing the sheath 264 to conform to the environment as well as gravity to create a shape that conforms to the anatomy of the individual wearing the urine collection device 202 thus giving the urine collection device 202 an adjustable length. Meanwhile, the upper ring 278 provides some rigidity to the urine collection device 202, thereby inhibiting leakage and patient injuries that the sheath 264, by itself, cannot prevent. In an embodiment, the upper ring 278 may be connected to the sheath 264. In such an embodiment, the upper ring 278 may be connected to the sheath 264 along with the sump 266 thereby increasing the attachment strength between the sheath 264 and the sump 266 or the upper ring 278 may indirectly connect the sump 266 to the sheath 264 (e.g., only the upper ring 278 is directly connected to the sheath 264).

In an embodiment, the upper ring 278 may include a writeable area that is configured to receiving one or more markings thereon, for example, from a pencil or marker. The writeable area of the upper ring 278 may allow medical practitioners to indicate when the urine collection device 202 was disposed on the individual or to indicate other useful information. In an embodiment, the upper ring 278 may include a material that exhibits a color change at or around a certain time after being exposed to air which allows the upper ring 278 to indicate how long the urine collection device 202 has been in use.

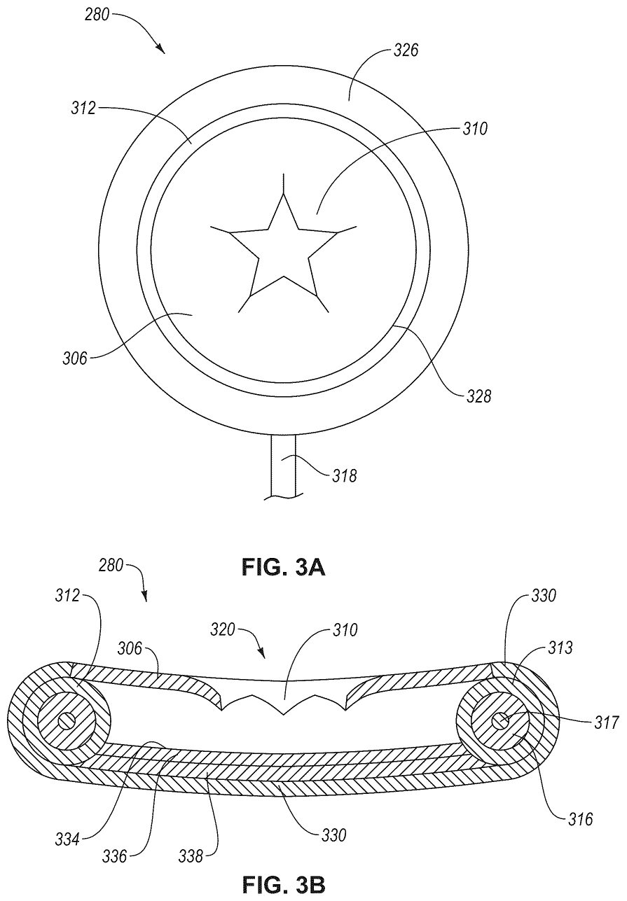

Referring to FIGS. 3A and 3B, an embodiment of a urine collection device 280 includes a chamber assembly 312. The chamber assembly 312 includes a thin layer of wicking material 313 and a porous material 316. The porous material 316 is configured to form a continuous ring-like chamber 317 in which urine can be collected for transport. The wicking material 313 can be disposed about the porous material 316. The chamber 317 has a port for receiving a tube 318 so that urine collected within the chamber 317 can be transported from the chamber 317 by being drawn from the chamber 317 when a partial vacuum is applied within the chamber 317 via the received tube 318. The received tube 318 can extend within the chamber 317. The chamber assembly 312 is dimensioned and configured to define a perimeter 328. The perimeter 328 can define an opening. An attachment interface 306 comprising a plurality of flaps 310 can extend from the perimeter 328 of the chamber assembly 312 through which the head of a penis can be inserted. The attachment interface 306 can be substantially similar to the attachment interfaces 106 and 206 discussed above.

In another embodiment (not shown), the porous material is configured to form a discontinuous C-shaped chamber in which urine can be collected for transport, with opposing portions of the chamber assembly being sufficiently adjacent as to define an opening through which the head of a penis can be inserted.

As illustrated in FIG. 3B, a flexible sheet of impermeable material 330 is so attached to the chamber assembly 312 to cover one side of the opening formed by the perimeter 328 and thereby provide a receptacle 320 for receiving the head of an inserted penis. Urine flowing into the receptacle 320 from the penis can be drawn through the wicking material 313 and the porous material 316 into the chamber 317 when a partial vacuum is applied within the chamber 317 via the tube 318.

In an embodiment, the sheet of impermeable material 330 is so dimensioned in relation to the breadth of the perimeter 328 as to extend sufficiently away from the chamber assembly 312 to provide adequate space in the receptacle to receive the head of the penis. The attachment interface 306 and the impermeable material 330 can form a volume to house the penis. The layer of impermeable material 330 further covers at least the exterior sides of the chamber assembly 312. The layer of impermeable material 330 can cover a portion of the interior sides of the chamber assembly 312.

The layer of impermeable material 330 can be attached to the chamber assembly 312 by an adhesive material. In another embodiment (not shown), retainer clips or other fasteners attach the impermeable material 330 to the chamber assembly 312. The impermeable material 330 can be integrally formed with the chamber assembly 312. Likewise, the attachment interface 306 can be attached to the chamber assembly 312 by an adhesive or other fastener. The attachment interface 306 can be integrally formed from the chamber assembly 312.

In an embodiment, the urine collection device 280 includes a cushion 334, which is disposed adjacent the impermeable material 330 for receiving the head of an inserted penis. The cushion 334 is so disposed over the layer of impermeable material 330 as to contact the wicking material 313 of the chamber assembly 312. The cushion 334 includes a layer of wicking material 336, such as medical gauze, disposed over a bed of porous material 338.

In an embodiment, the porous material 316 is provided as a web of a spun plastic material, such as nylon or polyester. In an embodiment, the chamber 317 is formed by folding together opposite sides of a web of spun plastic material, whereby the chamber 317 does not necessarily have a closed cross-section as shown in FIG. 2. In FIGS. 3A and 3B, the relative dimensions of the various components are not necessarily shown to scale.

In operation, when a man's penis is inserted through the flaps 310 of the attachment interface 306, expelled urine can flow between the layer of porous material 338 and the sheet of impermeable material 330 and into the chamber 317 and thence to the outlet tube 318. The urine collection device 280 can thus advantageously capture urine as it flows against gravity without having to attach a catheter to the penis. The attachment interface 306 can further prevent urine from escaping the urine collection device 280 and can direct urine toward the chamber 317.

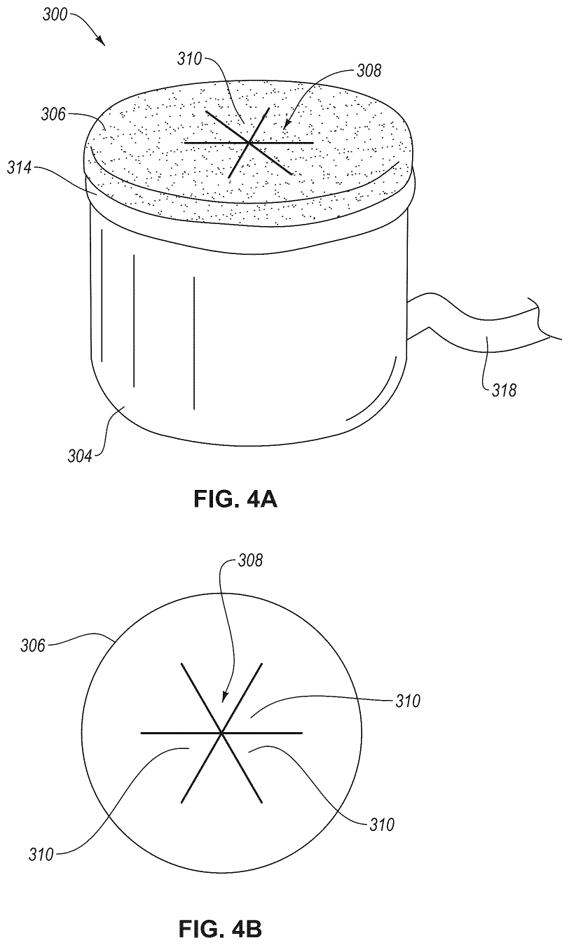

FIG. 4A is an isometric view of a urine collection device 300 (such as a male external catheter), according to an embodiment. The urine collection device 300 can include a receptacle 304 configured to collect urine from a subject. The receptacle 304 can be formed from silicone or other polymer materials. The urine collection device 300 can further include an attachment interface 306 configured to releasably couple to the subject. The attachment interface 306 can be integrally formed with the receptacle 304 or can be a separately formed unit that is coupled to the receptacle 304. For example, the attachment interface 306 can be configured to releasably attach to a penis. The attachment interface 306 can include a plurality of flaps 310 that are formed by one or more slits on the attachment interface 306. The plurality of flaps 310 can form an opening 308 that reveals the interior volume of the receptacle 304. The flaps 310 can be made from a flexible, resilient, and/or elastic material that is configured to retain its shape, such as a polymer material. For example, the flaps can be made from plastics such as polyethylene or polypropylene or from elastomers such as rubber. The flaps 310 can be configured to retain their position after being deformed. In this manner, the flaps 310 can generate an elastic tension against the subject's penis. Thus, a subject's penis can be brought in fluid communication with the interior volume of the receptacle 304 via opening 308. In some embodiments, the urine collection device 300 can include a wicking material 314 positioned proximate the attachment interface 306 within the interior volume of the receptacle 304. The wicking material 314 can be configured to draw fluid away from the opening 308 and the subject's body. For example, the wicking material 314 can be positioned in the V-shaped gaps between the flaps 310 and the subject's penis.

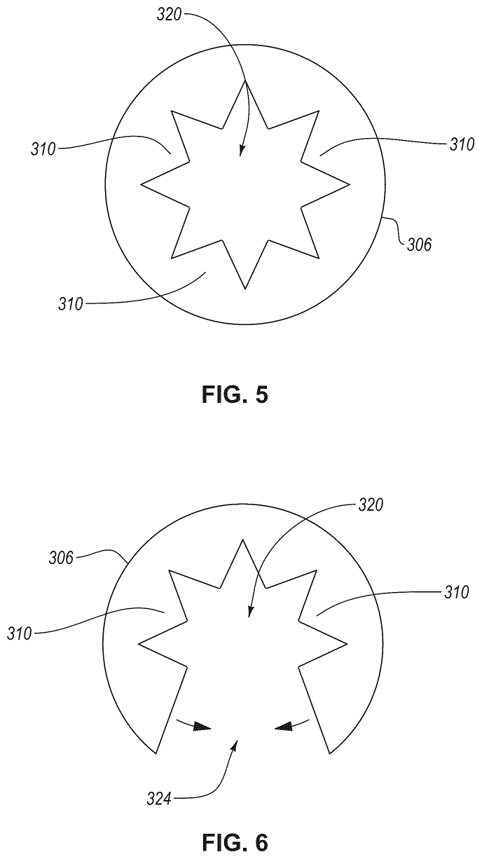

In some embodiments, the internal volume of the receptacle 304 defines an outlet that is in fluid communication with a conduit or tube 318. The tube 318 can be in fluid communication with a vacuum pump configured to draw a vacuum in the receptacle to suck any collected fluid out of the receptacle 304. Further details of the attachment interface 306 are provided below with reference to FIGS. 4 and 5.

FIG. 4B is a top view of an example attachment interface 306, according to an embodiment. The attachment interface 306 can include multiple flaps 310 that are formed by slits in the attachment interface 306. The slits allow the flaps to deform, bend, or fold to reveal the opening 308 into the receptacle 304. As shown in FIG. 4B, the slits can be arranged in a star-like pattern. In some embodiments, the flaps 310 are biased toward a neutral or flat position that is in-plane with the attachment interface 306, such that the opening 308 is covered when the flaps 310 are in their natural or unbiased position.

In some embodiments, the attachment interface 306 can include spun plastic. The attachment interface 306 can include multiple layers. In some embodiments, the attachment interface 306 includes an inner layer exposed to the internal volume of the receptacle 304. The inner layer can include a wicking material. The attachment interface 306 can include an outer layer exposed to the outside environment. The outer layer can include a fluid impermeable layer. A middle layer can be disposed between the inner layer and the outer layer. The inner layer can include spun plastic. The flaps 310 can be made from a flexible material that is configured to retain its shape. The flaps 310 can be configured to retain their position after being deformed. In this manner, the flaps 310 can generate an elastic tension against the subject's penis. In some embodiments, the flaps 310 can form a partial or complete seal against the subject's penis. For example, when the subject's penis is inserted through the opening 308. The flaps 310 bias against or press against the shaft of the penis to improve coupling between the receptacle 304 and the penis and to also generate a partial seal between the receptacle and the penis. In some embodiments, the flaps 310 entirely converge such that the opening 308 is entirely covered when the flaps are un-deformed. When the penis is positioned in the opening 308 and the flaps 310 are bent around the shaft of the penis, the shape of the flaps 310 naturally resist the penis exiting the attachment interface 306. In this way, the flaps 310 act as a type of one-way trap, allowing the penis to easily enter the opening 308, while resisting removal of the penis from the opening. However, removal of the penis from the attachment interface 306 can be easily performed and will not result is discomfort or injury to the subject.

FIG. 5 is a top view of an example attachment interface 306, according to an embodiment. The attachment interface 306 illustrated in FIG. 5 can be substantially similar to the attachment interface 306 disclosed with reference to FIG. 4B. The attachment interface 306 can include multiple flaps 310 that are formed by slits or cut-outs in the attachment interface 306. As shown in FIG. 5, a star-like cut-out can define an opening 320 into the receptacle 304. In some embodiments, the flaps 310 are biased toward a neutral or flat position. When the flaps 310 are in the neutral or unbiased state the opening 320 can be at a minimum diameter, which is expanded when the flaps 310 are deformed. In the embodiment shown in FIG. 5, the opening 320 remains at least partially exposed even when the flaps are in their unbiased position.

FIG. 6 illustrates an embodiment of an attachment interface 306 in which two ends of the attachment interface 306 are separated to form a gap 324, according to an embodiment. The gap 324 can be configured to allow a penis to enter the opening 320 through the gap 324. In other words, instead of having the penis enter the opening 320 by inserting the penis passed the flaps 310, the attachment interface 306 can wrap around the penis by permitting passage of the penis into the attachment interface 306. In an open state (i.e., with the gap 324 expanded) the attachment interface 306 can easily be wrapped around the penis, and in a closed state (i.e., with the gap closed or contracted) the penis can be secured within the attachment interface 306. Although the flaps shown herein are generally triangular in shape, it will be understood the other shapes are also possible, for example, the flaps can be semi-circular or rectangular.

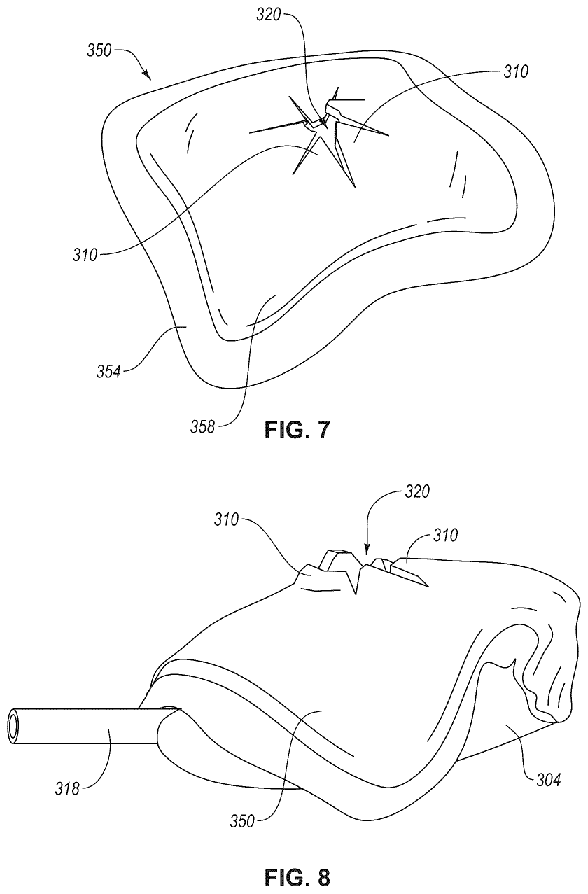

FIG. 7 illustrates a bandage 350. The bandage 350 can be an attachment interface and can be substantially similar to the attachment interfaces discussed herein, such as the attachment interfaces 306 of FIGS. 3-6, according to an embodiment. The bandage 350 can be configured to operationally couple with a urine collection device, such as with the receptacle 304 of urine collection device 300 discussed above. The bandage 350 can be formed of a silicone contact layer 354 and can include a wicking material 358. In some embodiments, the bandage 350 can comprise a foam dressing. The bandage 350 can include several slits or cut-outs that form a plurality of flaps 310. For example, the bandage 350 can include flaps 310 similar to the flaps discussed above with reference to the attachment interfaces 306 of FIGS. 3-6. In some embodiments, the bandage 350 includes an inner layer configured to directly contact the receptacle 304. The inner layer can include a wicking material and/or a tacky material, such as a silicone contact layer. The bandage 350 can include an outer layer exposed to the outside environment and configured to come into contact with the subject's body. The outer layer can include a fluid impermeable layer. A middle layer can be disposed between the inner layer and the outer layer. The inner layer can include spun plastic.

FIG. 8 illustrates the bandage 350 coupled to a receptacle 304 of a urine collection device, according to an embodiment. In some embodiments, the bandage 350 can include a sticky or tacky material, such as a silicone contact layer 354. For example, a major surface of the bandage 350 can include a tacky material that is capable of adhering to the receptacle 304. A second major surface of the bandage, opposite, the sticky side of the bandage 350 can include a soft material, such as polyurethane foam, intended to be comfortable against the subject's skin. In some embodiments, the bandage 350 does not use an adhesive to attach to the receptacle 304. In some embodiments, an adhesive is used to couple the bandage 350 to the receptacle 304. The hole 320 in the bandage 350 can be positioned adjacent a hole (not shown in FIG. 8) in the receptacle 304, such that the opening 320 is aligned with and in fluid communication with the interior of the receptacle 304 and further in fluid communication with the vacuum tube 318.

In some embodiments, the bandage 350 is coupled to a urine collection device, such as the urine collection devices of FIGS. 1-3. In this manner, a urine collection device could include multiple layers of flaps. For example, a first layer of flaps could be present on a wall of the receptacle 304 and a second layer of flaps could be present on the bandage 350. The layers of flaps can be oriented relative to each other such that the flaps 310 are in line with one another, or the layers can be off-set such that the flaps 310 of the bandage 350 are rotationally off-set from the flaps of the receptacle. In some embodiments, the flaps of each layer are different in shape and/or size. In some embodiments, the bandage 350 include multiple layers of flaps 310.

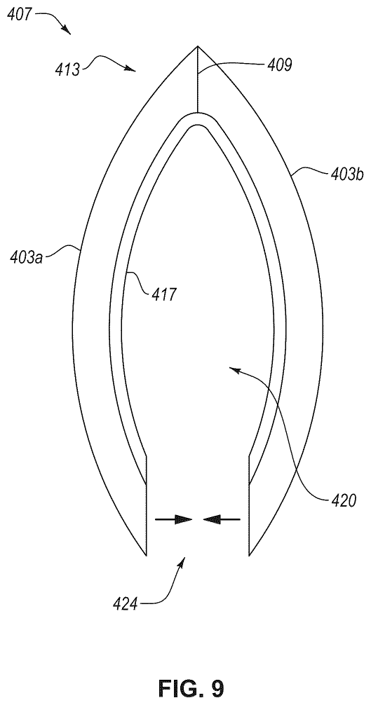

FIG. 9 illustrates an attachment mechanism or attachment interface 407 for attaching a urine collection device to a subject's penis, according to an embodiment. The attachment interface 407 is referred to herein as a clamshell, however, it will be understood that this term does not necessarily assign any properties, characteristics, or limitations onto the attachment mechanism 407. The clamshell 407 can include a first half 403a and a second half 403b. The first half 403a can be substantially similar or identical to the second half 403b, such that the clamshell 407 is symmetrical about an axis. The first half 403a can be connected to the second half 403b via a pivot point 409 at a distal end 413 of the clamshell 407. The pivot point 409 can include a hinge, joint, axle, pin, spring or any other mechanism capable of allowing relative motion between the first half 403a and the second half 403b. In some embodiments, the clamshell 407 is flexible such that the first half 403a and the second half 403b can be bent toward or away from one another. The clamshell 407 can be biased toward a closed position (i.e., where the first half 403a and second half 403b are closest at a proximal end of the clamshell 407).

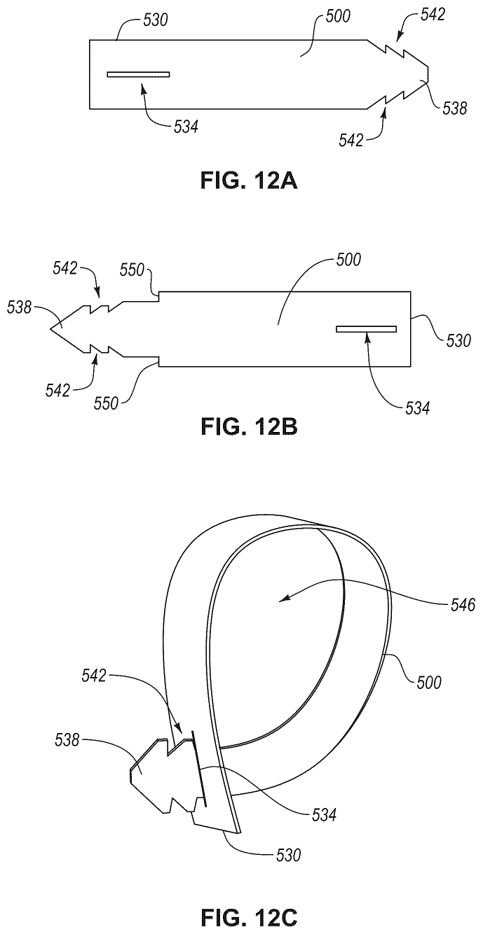

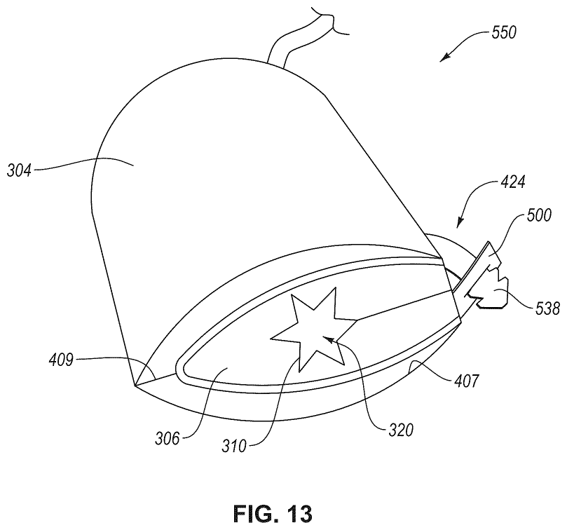

In some embodiments, the clamshell 407 can include a vacuum tube and wicking layers such as those discussed above with reference to the urine collection device 280 of FIGS. 3A and 3B. For example, the clamshell 407 can include a layer of wicking material, such as a matrix of spun plastic that at least partially surrounding a vacuum tube to collect and transport urine. The clamshell 407 can further be in fluid communication with an outlet tube attached to a sump of the clamshell 407. In some embodiments, the clamshell 407 can include a securing feature (not shown in FIG. 9) configured to secure the proximal end of the first half 403a to the proximal end of the second half 403b. In other words, the securing feature can be configured to secure the clamshell in the closed position. The securing feature can include Velcro, an adhesive, or a strip, such as the strip 500 discussed below with reference to FIGS. 12A-13.

In operation, the clamshell 407 can be position around a penis, with the penis positioned between the first half 403a and the second half 403b in the opening 420. When in the closed position, the clamshell 407 can be frictionally secured to the penis. In some embodiments, the clamshell 407 can be configured to secure a base of the urine collection device to the base of the penis. The clamshell 407 can be configured to clamp, constrict, or squeeze the penis sufficient to secure the clamshell 407 to the penis, while still allowing urine flow. In some examples, the clamshell 407 can include different levels of constriction or clamping. For example, the degree of clamping or the pressure applied onto the penis can be increased to improve the fit or attachment onto the penis. The clamshell 407 can include an inner layer 417 that is tacky or sticky to enhance the friction securement between the clamshell 407 and the penis. In some embodiments, the inner layer 417 includes a rugged surface with enhanced friction. For example, the inner layer 417 can include spun plastic. In an embodiment, the inner layer 417 includes flaps similar to the flaps 310.

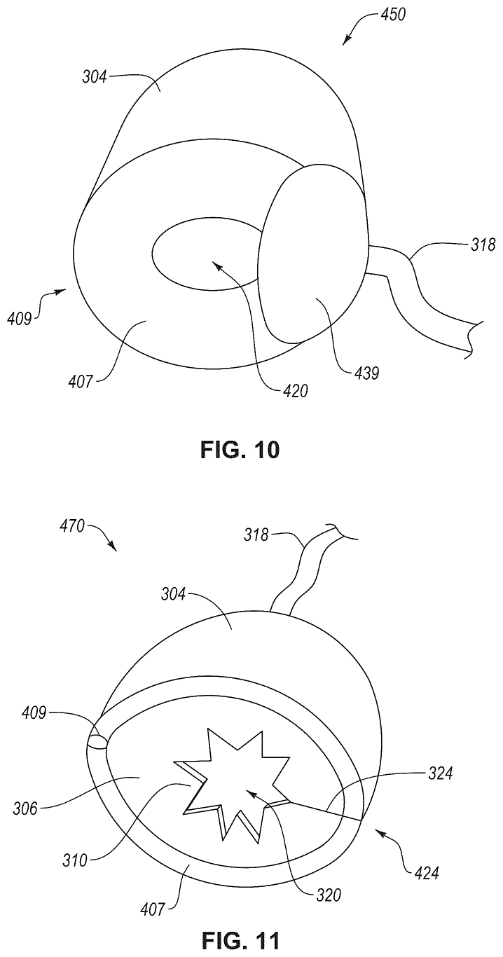

FIG. 10 illustrates a clamshell 407 functionally coupled with the receptacle 304 of a urine collection device 450, according to an embodiment. The urine collection device 450 can be substantially similar to the urine collection device 300 discussed above with reference to FIG. 4A. For example, the urine collection device 450 can include a receptacle 304 in fluid communication with a vacuum tube 318. The clamshell 407 can be integrally formed with the receptacle 304 or can be a separate and distinct piece configured to attach and form a seal with the receptacle 304. The urine collection device 450 can include a sump 439 configured to collect urine to be suctioned from the receptacle 304. In some embodiments, the sump 439 is coupled to the clamshell 407.

FIG. 11 illustrates a urine collection device 470 including an attachment interface 306 and a clamshell 407 operationally coupled to the receptacle 304, according to an embodiment. The attachment interface 306 and clamshell 407 can be substantially similar to the attachment interfaces and clamshells discussed herein. In some embodiments, the flaps 310 of the attachment interface 306 can extend from a perimeter of the clamshell 407. The flaps 310 can be integrally formed from the inner sidewall of the clamshell 407. A gap 324 in the attachment interface 306 can coincide with the gap 424 in the clamshell 407. In this manner, the features and improvements of the attachment interfaces can be combined with the features and improvements of the clamshell 407 to produce enhanced attachment methodologies of the urine collection device. The gaps 324 and 424 in the urine collection device 470 could be closed and secured using any securement method, such as Velcro, adhesive, knots, or the strips disclosed herein.

FIG. 12A illustrates a strip 500, according to an embodiment. The strip 500 can be considered an attachment interface and can be used as a closing, tightening, or constricting device. For example, the strip 500 can be used in conjunction with the attachment interfaces discussed herein to further aid is securing the urine collection device to the penis. In some embodiments, the strip 500 can be used to wrap directly around the penis and can, in some cases, be used to constrict the penis to restrict urine flow. The strip 500 can be formed from a piece of coarse spun plastic. The strip 500 can include a tip 538 formed on a first end of the strip 500 and a slot 534 formed on a second end of the strip 500, opposite the first end. The tip 538 can include one or more catches or jagged section 542 configured to pass through the slot 534 in one direction and catch or snag on the slot 534 in a second direction. In this manner, the strip 500 can be a pull-through strap configured to secure to itself via friction.

FIG. 12B illustrates the strip 500 including a shelf or stop 550 configured to prevent the tip 538 from entering any further into the slot 534. FIG. 12C illustrates the strip 500 with the tip 538 engaged in the slot 534. As illustrated, when the tip 538 is fed through the slot 534, the strip 500 forms a loop or noose 546. In some embodiments, the loop formed by the strip 500 can act as an attachment or securement mechanism. The catches 542 can help prevent the tip 538 from backing out of the slot 534, and the shelf 550 can prevent the strip 500 from being pulled too tight. In some embodiments, the strip 500 is formed of a coarse material. The course nature of the strip 500 can aid the tip 538 from slipping out of the slot 534. The coarseness of the strip 500 can also help keep the strip 500 in place against the user's skin and thereby reducing abrasive forces against the user's skin. The strip 500 can be used in conjunction with any of the attachment interfaces or clamshell devices disclosed herein. For example, the strip 500 can be used to close or tighten the gap 324 of the attachment interface 306 and the gap 424 of the clamshell 407.

FIG. 13 is a bottom isometric view of a urine collection device 550 having multiple attachment features as described herein, according to an embodiment. The urine collection device 550 of FIG. 13 can include an attachment interface 306 and clamshell 407 for removably securing the urine collection device to the penis. Further, a strip 500, similar to the strips disclosed herein, can be positioned on either ends of the attachment interface 306 and/or the clamshell 407 such that the clamshell 407 and the attachment interface 306 can be closed around the penis when the strip 500 is pulled through itself, thereby closing the gap 424. The strip 500 can be integrally formed with either of the clamshell 407 or attachment interface 306, such that cinching the strip 500 also causes the clamshell 407 or attachment interface 306 to tighten around the penis. In an embodiment, the strip 500 is positioned along an inner circumference of the receptacle 304. In an embodiment, the strip 500 wraps around the circumference of the receptacle 304. Thus, it will be understood that each of the attachment interfaces described herein (e.g., attachment interface 306, clamshell 407, and strip 500) can be used individually or in combination to improve attachment of the urine collection device to the penis.

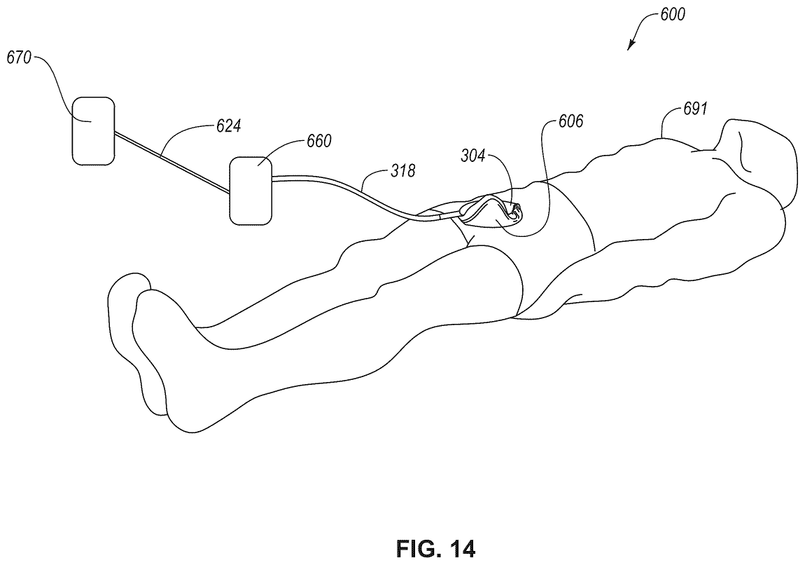

FIG. 14 is a schematic illustration of a urine collecting system 600 disposed on the body of a subject 691, according to an embodiment. The urine collecting system 600 can utilize any of the urine collection devices and attachment interfaces disclosed herein. The urine collecting system 600 can include a receptacle 304 to collect urine and an attachment interface 606. The attachment interface 606 can be the same or similar in structure and/or function to any of the attachment interfaces described herein, such as attachment interfaces 106, 306, 350, 407, 500, and any combination thereof. The urine collecting system 600 can include an external receptacle 660 and a vacuum source 670. The external receptacle 660 can be a temporary storage location for urine drawn from the receptacle 304. In an embodiment, the vacuum source 670 can assist and/or provide the pressure differential needed to draw fluid voided from the urethral opening of a user into the receptacle 104, and from the receptacle 304 into the external receptacle 660. The vacuum source 670 can be fluidly coupled to the external receptacle 660 via a vacuum line 624 such that urine is drawn from the external receptacle 660 via the vacuum line 624. As a result of the decrease in pressure within the external receptacle 660 caused by the drawing of fluid out of the external receptacle 660, liquid and/or gaseous fluid can be drawn from the receptacle 304, through the tube 318, and into the external receptacle 660. In an embodiment, the vacuum source 670 can apply sufficient suction to capture all or substantially all of the urine voided by a user in a variety of positions (e.g., when a user is lying on his side).

In an embodiment, the vacuum source 670 can be a pump that is readily available, inexpensive, relatively quiet, and/or configured to run continuously. For example, the vacuum source 670 can be a pump. In an embodiment, the vacuum source 670 can be powered by electrical AC or DC power. For example, in mobile applications when the user is away from an AC power source, such as when the user is using the urine collecting system 600 during transportation via a wheel chair or motor vehicle, the vacuum source 670 can be powered by DC power. One suitable non-limiting example of a pump that can be used is the DryDoc Vacuum Station, available from PureWick, Inc.

In use, as shown in FIG. 14, the urine collecting system 600 can be positioned such that the attachment interface 606 encloses a portion of the penis of the subject 491. For example, the attachment interface 606 can be positioned such that the urethral opening of the user 691 is within an internal volume of the receptacle 304.

While various embodiments of the urine collecting system, methods and devices have been described above, it should be understood that they have been presented by way of example only, and not limitation. Where methods and steps described above indicate certain events occurring in certain order, those of ordinary skill in the art having the benefit of this disclosure would recognize that the ordering of certain steps may be modified and such modifications are in accordance with the variations of the invention. Additionally, certain of the steps may be performed concurrently in a parallel process when possible, as well as performed sequentially as described above. The embodiments have been particularly shown and described, but it will be understood that various changes in form and details may be made.

For example, although various embodiments have been described as having particular features and/or combinations of components, other embodiments are possible having any combination or sub-combination of any features and/or components from any of the embodiments described herein. In addition, the specific configurations of the various components can also be varied. For example, the size and specific shape of the various components can be different than the embodiments shown, while still providing the functions as described herein.

1. (canceled)

2. A male urine collection device for collection of urine discharged from a subject, the male urine collection device comprising:
a fluid impermeable barrier including a first panel and a second panel secured to the first panel, the fluid impermeable barrier defining a chamber between the first panel and the second panel and having an outlet at a distal end region, wherein the second panel is substantially flat and at least partially defines an opening at a proximal end region sized and dimensioned to receive a penis therethrough when the penis is inserted through the opening into the chamber;
one or more permeable members positioned within the chamber between the first panel and the second panel; and
an attachment interface disposed in the opening and positioned and configured to encircle the penis when the penis is inserted through the opening.

3. The male urine collection device of claim 2, wherein the attachment interface includes one or more flaps shaped and configured to allow the penis to be inserted through the opening and resist the penis exiting the opening.

4. The male urine collection device of claim 3, wherein the one or more flaps include a material configured to retain a shape and a position of the one or more flaps after deformation effective to generate elastic tension against the penis.

5. The male urine collection device of claim 3, wherein the one or more flaps are configured to bias against the penis when the penis is inserted through the opening.

6. The male urine collection device of claim 3, wherein the one or more flaps include a plurality of flaps that entirely converge such that the opening is completely covered when the plurality of flaps are undeformed without the penis inserted through the opening.

7. The male urine collection device of claim 3, wherein the one or more flaps define an additional opening positioned within the opening of the second panel of the fluid impermeable barrier.

8. The male urine collection device of claim 3, wherein the one or more flaps include a first layer of flaps and a second layer of flaps rotationally offset from the flaps of the first layer.

9. The male urine collection device of claim 2, wherein the attachment interface includes a tacky, non-adhesive material.

10. The male urine collection device of claim 2, wherein the attachment interface is substantially flat with the second panel.

11. A urine collection system, comprising:
a male urine collection device including:
a fluid impermeable barrier including a first panel and a second panel secured to the first panel, the fluid impermeable barrier defining a chamber between the first panel and the second panel and having an outlet at a distal end region, wherein the second panel is substantially flat and at least partially defines an opening at a proximal end region sized and dimensioned to receive a penis therethrough when the penis is inserted through the opening into the chamber;
one or more permeable members positioned within the chamber between the first panel and the second panel; and
an attachment interface disposed in the opening and positioned and configured to encircle the penis when the penis is inserted through the opening;
an external receptacle in fluid communication with the outlet of the male urine collection device; and
a vacuum source configured to apply suction and draw urine from the chamber of the male urine collection device, through the outlet, and into the external receptacle.

12. The urine collection system of claim 11, wherein the attachment interface includes one or more flaps shaped and configured to allow the penis to be inserted through the opening and resist the penis exiting the opening.

13. The urine collection system of claim 12, wherein the one or more flaps include a material configured to retain a shape and a position of the one or more flaps after deformation effective to generate elastic tension against the penis.

14. The urine collection system of claim 12, wherein the one or more flaps are configured to bias against the penis when the penis is inserted through the opening.

15. The urine collection system of claim 12, wherein the one or more flaps include a plurality of flaps that entirely converge such that the opening is completely covered when the plurality of flaps are undeformed without the penis inserted through the opening.

16. The urine collection system of claim 12, wherein the one or more flaps define an additional opening positioned within the opening of the second panel of the fluid impermeable barrier.

17. The urine collection system of claim 12, wherein the one or more flaps include a first layer of flaps and a second layer of flaps rotationally offset from the flaps of the first layer.

18. The urine collection system of claim 11, wherein the attachment interface includes a tacky, non-adhesive material.

19. The urine collection system of claim 11, wherein the attachment interface is substantially flat with the second panel.

20. A method of collecting urine from a subject, the method comprising:
inserting a penis through an opening in a urine collection device and at least partially into a chamber of the urine collection device with an attachment interface that is disposed in the opening encircling the penis as the penis is inserted through the opening, the urine collection device including:
a fluid impermeable barrier including a first panel and a second panel secured to the first panel, the fluid impermeable barrier defining the chamber between the first panel and the second panel, wherein the second panel is substantially flat and at least partially defines the opening at a proximal end region of the urine collection device; and
one or more permeable members positioned within the chamber between the first panel and the second panel;
collecting urine voided by the subject in the chamber of the urine collection device;
applying suction to an outlet at a distal end region of the urine collection device with a vacuum source to draw urine from the chamber through the outlet and into an external receptacle in fluid communication with the outlet.

21. The method of claim 20, wherein the attachment interface includes one or more flaps shaped that allow the penis to be inserted through the opening and resist the penis exiting the opening with at least one of elastic tension against the penis or a bias against the penis.

22. The method of claim 21, wherein the one or more flaps include a plurality of flaps that completely cover the opening before the penis is inserted through the opening.

23. The method of claim 21, wherein the one or more flaps define an additional opening positioned within the opening of the second panel of the fluid impermeable barrier.

24. The method of claim 21, wherein the one or more flaps include a first layer of flaps and a second layer of flaps rotationally offset from the flaps of the first layer.

25. The method of claim 20, wherein the attachment interface is substantially flat with the second panel.
